(19) United States
(12) Patent Application Publication
(10) Pub. No.: US 2026/0049046 A1
(43) Pub. Date: Feb. 19, 2026

(54) METHOD OF PRODUCING FLUOROOLEFIN

(71) Applicant: AGC INC., Tokyo (JP)

(72) Inventors: Hikaru IWASAKI, Tokyo (JP); Taku YAMADA, Tokyo (JP); Eri SAKAKIBARA, Tokyo (JP)

(73) Assignee: AGC INC., Tokyo (JP)

(21) Appl. No.: 19/369,109

(22) Filed: Oct. 24, 2025

Related U.S. Application Data
(63) Continuation of application No. PCT/JP2024/014304, filed on Apr. 8, 2024.

(30) Foreign Application Priority Data
Apr. 26, 2023 (JP) 2023-072721

(51) Int. Cl. C07C 17/23

(52) U.S. Cl. CPC C07C 17/23

(57) ABSTRACT

A method of producing fluoroolefin includes contacting a fluorocarbon represented by Formula (1): CX¹X²F—CX³X⁴H with an alumina-containing catalyst to produce a fluoroolefin represented by Formula (2): CX¹X²═CX³X⁴, in which the alumina-containing catalyst satisfies at least one of the following conditions (I) or (II). (I) A total content of alkali metal elements and alkaline earth metal elements is 100 ppm by mass or less. (II) A Si content is 1000 ppm by mass or less.

### CROSS-REFERENCE TO RELATED APPLICATIONS

This application is a Continuation of International Application No. PCT/JP2024/014304, filed Apr. 8, 2024, which claims priority to Japanese Patent Application No. 2023-072721 filed Apr. 26, 2023. Each of the above applications is hereby expressly incorporated by reference, in its entirety, into the present application.

### TECHNICAL FIELD

This disclosure relates to a method of producing a fluoroolefin.

### BACKGROUND ART

In recent years, fluoroolefins have attracted attention as compounds with low global warming potential, and their production methods are being investigated.

For example, Non-Patent Document 1 describes a method for obtaining trifluoroethylene using α-alumina, γ-alumina, θ-alumina or the like in the dehydrofluorination reaction of 1,1,1,2-tetrafluoroethane.

### PRIOR ART LITERATURE

### Non-Patent Document

Non-Patent Document 1: Catalysis Letters 2015, Vol. 145, pp. 654-661

### SUMMARY OF INVENTION

However, as shown in FIG. 1 of Non-Patent Document 1, in a case in which various kinds of aluminas were used as catalysts, there were instances in which the conversion rate of the reaction was low.

One embodiment in the present disclosure aims to provide a method of producing fluoroolefins with a higher conversion rate than conventional methods.

### Solution to Problem

The present disclosure includes the following aspects.

<1> A method of producing a fluoroolefin, the method including contacting a fluorocarbon represented by the following Formula (1) with an alumina-containing catalyst to produce a fluoroolefin represented by the following Formula (2),

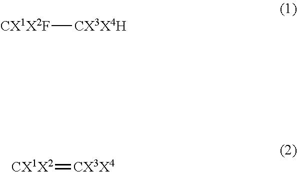

in which, in Formulas (1) and (2), X¹, X², X³ and X⁴ each independently represent a hydrogen atom or a fluorine atom, provided that at least one of X¹, X², X³ or X⁴ is a fluorine atom, and
in which the alumina-containing catalyst satisfies at least one of the following (I) or (II):
(I) a total content of alkali metal elements and alkaline earth metal elements is 100 ppm by mass or less;
(II) a Si content is 1000 ppm by mass or less.

<2> The method of producing a fluoroolefin according to <1>, in which the alumina contained in the alumina-containing catalyst includes α-alumina.

<3> The method of producing a fluoroolefin according to <1> or <2>, in which the fluorocarbon is at least one selected from the group consisting of 1,1,1-trifluoroethane, 1,1,2-trifluoroethane, 1,1,2,2-tetrafluoroethane, and 1,1,1,2-tetrafluoroethane.

<4> The method of producing a fluoroolefin according to any one of <1> to <3>, in which the fluoroolefin is at least one selected from the group consisting of 1,2-difluoroethylene, 1,1-difluoroethylene, and trifluoroethylene.

<5> The method of producing a fluoroolefin according to any one of <1> to <4>, in which the fluorocarbon is 1,1,1,2-tetrafluoroethane, and the fluoroolefin is trifluoroethylene.

<6> The method of producing a fluoroolefin according to any one of <1> to <5>, in which the fluorocarbon and the alumina-containing catalyst are contacted at a temperature of from 300 to 800° C.

<7> The method of producing a fluoroolefin according to any one of <1> to <6>, in which:
the fluorocarbon and the alumina-containing catalyst are contacted in the presence of an inert gas; and
the inert gas is at least one selected from the group consisting of nitrogen, helium, argon, octafluorocyclobutane, and carbon dioxide.

<8> The method of producing a fluoroolefin according to any one of <1> to <7>, further including drying the alumina-containing catalyst before contacting the fluorocarbon with the alumina-containing catalyst.

<9> The method of producing fluoroolefins according to any one of <1> to <8>, in which:
the fluorocarbon and the alumina-containing catalyst are contacted in a gas phase in the presence of water; and
a concentration of the water is less than 500 ppm by mass with respect to a total amount of a feed gas containing the fluorocarbon.

### Advantageous Effects of Invention

According to the present disclosure, it is possible to provide a method of producing fluoroolefins with a higher conversion rate than conventional methods.

### DESCRIPTION OF EMBODIMENTS

In the present disclosure, numerical ranges indicated using “to” refer to ranges that include the numerical values before and after “to” as the minimum and maximum values, respectively.

In numerical ranges described in a stepwise manner in the present disclosure, the upper limit value or lower limit value of one range may be replaced with the upper limit value or lower limit value of another stepwise-described range. Additionally, in the numerical ranges described in the present disclosure, the upper limit value or lower limit value of one range may be replaced with a value shown in the examples.

In the present disclosure, a combination of two or more preferred aspects is a more preferred aspect.

In the present disclosure, the amount of each component in a composition refers, unless otherwise specified, to the total amount of multiple substances corresponding to that component in a case in which multiple such substances are present in the composition.

### [Method of Producing Fluoroolefin]

A method of producing a fluoroolefin in the present disclosure includes contacting a fluorocarbon represented by the following Formula (1) with an alumina-containing catalyst to produce a fluoroolefin represented by the following Formula (2),

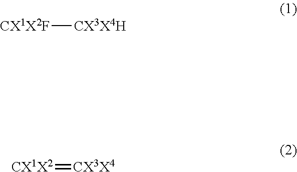

in which, in Formulas (1) and (2), X¹, X², X³ and X⁴ each independently represent a hydrogen atom or a fluorine atom, provided that at least one of X¹, X², X³ or X⁴ is a fluorine atom, and
in which the alumina-containing catalyst satisfies at least one of the following (I) or (II):
(I) a total content of alkali metal elements and alkaline earth metal elements is 100 ppm by mass or less;
(II) a Si content is 1000 ppm by mass or less.

The method of producing a fluoroolefin in the present disclosure achieves a higher conversion rate than conventional methods. The reasons for this are not clear, but are presumed to be as follows.

In the reaction from the fluorocarbon represented by Formula (1) to the fluoroolefin represented by Formula (2), hydrogen fluoride is generated. The generated hydrogen fluoride reacts with the fluoroolefin represented by Formula (2) to revert to the fluorocarbon represented by Formula (1). Because this reaction proceeds, the conversion rate of the fluorocarbon represented by Formula (1) has traditionally been very low.

Furthermore, as described in the above-mentioned Non-Patent Literature 1, in a case in which alumina was used as a catalyst, the conversion rate was sometimes low. Although the cause of this may be attributed to differences in the crystal structure of α-alumina, γ-alumina, and the like, other factors may also be responsible.

Accordingly, the present inventors conducted compositional analyses of various alumina-containing catalysts, and as a result, found that a content ratios of alkali metals and alkaline earth metals, as well as a content ratio of Si, in the alumina-containing catalysts affected the conversion rate. Through further studies, the inventors experimentally found that, when using an alumina-containing catalyst satisfying at least one of the above (I) and (II), the conversion rate increases.

### (Fluorocarbon Represented by Formula (1))

In the method of producing a fluoroolefin in the present disclosure, a fluorocarbon represented by the following Formula (1) is used as a raw material.

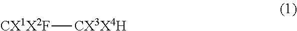

In Formula (1), X¹, X², X³ and X⁴ each independently represent a hydrogen atom or a fluorine atom, provided that at least one of X¹, X², X³ or X⁴ is a fluorine atom.

Examples of the fluorocarbon represented by Formula (1) include the following compounds.
CHF₂CH₃: 1,1-difluoroethane (HFC-152a)
CH₂FCH₂F: 1,2-difluoroethane (HFC-152)
CF₃CH₃: 1,1,1-trifluoroethane (HFC-143a)
CHF₂CH₂F: 1,1,2-trifluoroethane (HFC-143)
CF₃CH₂F: 1,1,1,2-tetrafluoroethane (HFC-134a)
CHF₂CHF₂: 1,1,2,2-Tetrafluoroethane (HFC-134)
CF₃CHF₂: 1,1,1,2,2-Pentafluoroethane (HFC-125)

Among these, the fluorocarbon represented by Formula (1) is preferably at least one selected from the group consisting of HFC-143a, HFC-143, HFC-134a, and HFC-134, from the viewpoint of reducing side reactions and suppressing the production of by-products. Furthermore, the fluorocarbon represented by Formula (1) is preferably HFC-134a, since one kind of fluoroolefin can be obtained with high selectivity

### (Fluoroolefin Represented by Formula (2))

The method of producing a fluoroolefin in the present disclosure produces a fluoroolefin represented by the following Formula (2) as the reaction product.

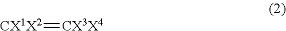

In Formula (2), X¹, X², X³ and X⁴ each independently represent a hydrogen atom or a fluorine atom, provided that at least one of X¹, X², X³ or X⁴ is a fluorine atom.

Examples of the Fluoroolefin represented by Formula (2) include the following compounds:
CHF═CH₂: Fluoroethylene
CF₂═CH₂: 1,1-Difluoroethylene (HFO-1132a)
CHF═CHF: 1,2-Difluoroethylene (HFO-1132(E), HFO-1132(Z))
CHF═CF₂: Trifluoroethylene (HFO-1123)
CF₂═CF₂: Tetrafluoroethylene

Among these, from the viewpoint of usefulness as a refrigerant composition, the fluoroolefin represented by Formula (2) is preferably at least one selected from the group consisting of HFO-1132, HFO-1132a, and HFO-1123.

In particular, in the method of producing a fluoroolefin in the present disclosure, from the viewpoint of allowing the reaction to proceed more selectively, it is preferable that the fluorocarbon is HFC-134a, and the fluoroolefin is HFO-1123.

In addition to the fluoroolefin represented by Formula (2), it is also possible to produce an olefin other than the fluoroolefin of Formula (2), and as the olefin other than Formula (2), ethylene may be included.

### (Alumina-Containing Catalyst)

In the method of producing a fluoroolefin in the present disclosure, a fluorocarbon represented by Formula (1) is contacted with an alumina-containing catalyst. The alumina-containing catalyst contacted with the fluorocarbon satisfies at least one of the following (I) or (II):
(I) a total content of alkali metal elements and alkaline earth metal elements is 100 ppm by mass or less;
(II) a Si content is 1000 ppm by mass or less.

In the above (I), the expression “100 ppm by mass or less” encompasses both a case in which a target element is contained in an amount of 100 ppm by mass or less, and a case in which the target element is not present (i.e., 0 ppm by mass). The same applies to cases in which only the upper limit value is specified for the content of other elements.

In the present disclosure, an alumina-containing catalyst refers to one that contains aluminum (Al) and oxygen (O) when subjected to elemental analysis by X-ray fluorescence analysis (XRF). The alumina-containing catalyst preferably contains Al in an amount of 1.0% by mass or more, but may contain 5.0% by mass or more, or 10% by mass or more.

Hereinafter, a content of each element in the catalyst is referred to as a value obtained by elemental analysis by XRF. XRF is performed using an X-ray fluorescence analyzer (e.g., a ZSX Primus II scanning X-ray fluorescence analyzer manufactured by Rigaku Corporation) to perform qualitative and quantitative analysis measurements. All elements present in the measurement area are measured, and the atomic weights of these elements and the atomic weight of a specific metal elements are each determined. Then, the atomic weights of all elements present in the measurement area and the atomic weight of a target element are each converted to mass. From these results, the percentage by mass of each element with respect to the total mass of all elements present in the measurement area is calculated.

In a case in which the alumina-containing catalyst is to be used after various treatments such as drying and activation, elemental analysis of the alumina-containing catalyst may be performed before or after the treatments.

Alumina contained in the alumina-containing catalyst may be a single type or a combination of two or more types.

Alumina is a dehydrated product of aluminum hydroxide, and its properties vary depending on the degree of dehydration and crystallinity. Examples of alumina contained in the alumina-containing catalyst include β-alumina, γ-alumina, θ-alumina, η-alumina, boehmite, and gibbsite, it is preferable that the catalyst contains α-alumina. Compared with other alumina structures, α-alumina is a high-temperature stable phase having a high degree of crystallinity. Although it has a small specific surface area, it is thermally stable and has high thermal conductivity. Furthermore, compared with other alumina structures, α-alumina has a higher conversion barrier from Al—O to Al—F in the presence of hydrogen fluoride. Therefore, using a catalyst containing α-alumina can suppress the formation of AlF₃, and more effectively prevent catalyst deactivation and a decrease in selectivity.

Although the reason why the selectivity is improved with α-alumina compared with γ-alumina is not clear, it is considered that this γ-alumina undergoes significant changes in crystal structure under high-temperature environments, and that changes in the bonding distance between alkali metal elements, alkaline earth metal elements, and Si and Al in the alumina-containing catalyst influence the selectivity.

In addition, since α-alumina has higher catalyst durability than γ-alumina, in a case in which a feed amount of raw material to the catalyst is large, a decrease in conversion rate during long-term production can be effectively suppressed. Therefore, by using a catalyst containing α-alumina, it becomes possible to increase the feed amount of raw material to the catalyst, which provides advantages in industrial production.

The presence of α-alumina in the alumina-containing catalyst can be confirmed by the diffraction pattern obtained using X-ray diffraction method, in other words, XRD (X-ray diffractometer). Commercially available XRD equipment can be used, such as “Smart Lab” manufactured by Rigaku Corporation. The presence of peaks at d=26.62, 35.21, 37.85, 43.43, 52.65, and 57.61 Å in the diffraction pattern indicates the presence of α-alumina. This analysis is to be performed on the alumina-containing catalyst immediately before contact with fluorocarbon, or on the alumina-containing catalyst in which the same state as that immediately before being brought into contact with the fluorocarbon is reproduced.

The alumina-containing catalyst may also contain aluminum oxide fluoride or aluminum fluoride, in which alumina is fluorinated.

The alumina-containing catalyst may also contain a compound other than alumina. Examples of the compound other than alumina include oxides other than alumina, such as chromium oxide, copper oxide, iron oxide, nickel oxide, magnesium oxide, zinc oxide, and zirconium oxide.

In particular, from the viewpoint of maintaining the durability of the alumina-containing catalyst, it is preferable that, in a diffraction pattern obtained by X-ray diffraction, among the peaks attributable to α-alumina, the diffraction intensity of a peak at d=43.43 Å is higher than the diffraction intensity of peaks attributable to compounds other than α-alumina, or that the diffraction pattern only has peaks attributable to α-alumina. In other words, it is preferable that the alumina-containing catalyst be primarily composed of α-alumina.

As described above, the peaks attributable to α-alumina are the peaks at d=26.62, 35.21, 37.85, 43.43, 52.65, and 57.61 Å.

In a case in which the alumina-containing catalyst contains α-alumina, a content of the α-alumina crystal structure may be 65% by mass or more, 70% by mass or more, 75% by mass or more, 80% by mass or more, 85% by mass or more, or even 100% by mass. The content of α-alumina crystal structures in an alumina-containing catalyst can be confirmed from the crystal structure obtained by XRD by performing Rietveld analysis. Specifically, peaks obtained by XRD measurement of the catalyst are compared with known peak models derived from respective alumina structures, and by performing Rietveld analysis, the mass ratio of each crystal structure is calculated.

A total content of alkali metal elements and alkaline earth metal elements in the alumina-containing catalyst is 100 ppm by mass or less, preferably 80 ppm by mass or less, more preferably 50 ppm by mass or less, and may be 0 ppm by mass (i.e., not present).

In a case in which a Si content in the alumina-containing catalyst is 1000 ppm by mass or less, the total content of alkali metal elements and alkaline earth metal elements in the alumina-containing catalyst may exceed 100 ppm by mass, but is preferably 100 ppm by mass or less.

A content of the alkali metal element in the alumina-containing catalyst is 100 ppm by mass or less, preferably 40 ppm by mass or less, more preferably 25 ppm by mass or less, and may be 0 ppm by mass (i.e., not contained). In a case in which the Si content in the alumina-containing catalyst is 1000 ppm by mass or less, the content of the alkali metal element in the alumina-containing catalyst may exceed 100 ppm by mass, but is preferably 100 ppm by mass or less.

Among alkali metal elements, examples of an element that are likely to be contained in alumina-containing catalysts include Na and K. A total content of Na and K in the alumina-containing catalysts is 100 ppm by mass or less, preferably 40 ppm by mass or less, more preferably 25 ppm by mass or less, and may even be 0 ppm by mass (i.e., not contained).

A content of the alkaline earth metal element in alumina-containing catalysts is 100 ppm by mass or less, preferably 40 ppm by mass or less, more preferably 25 ppm by mass or less, and may even be 0 ppm by mass (i.e., not contained). In a case in which the Si content in the alumina-containing catalyst is 1000 ppm by mass or less, the content of the alkaline earth metal element in the alumina-containing catalyst may exceed 100 ppm by mass, but is preferably 100 ppm by mass or less.

Among alkaline earth metal elements, examples of an element that are likely to be contained in alumina-containing catalysts include Mg and Ca. A total content of Mg and Ca in the alumina-containing catalyst is 100 ppm by mass or less, preferably 40 ppm by mass or less, more preferably 25 ppm by mass or less, and may be 0 ppm by mass (i.e., not contained).

The Si content in the alumina-containing catalyst is 1000 ppm by mass or less, preferably 800 ppm by mass or less, more preferably 600 ppm by mass or less, and may be 0 ppm by mass (i.e., not contained). In a case in which the alkaline earth metal element content in the alumina-containing catalyst is 100 ppm by mass or less, the Si content in the alumina-containing catalyst may exceed 1000 ppm by mass, but is preferably 1000 ppm by mass or less.

In addition to Al and O, the alumina-containing catalyst may contain other elements such as C, Fe, Zn, and Ga. A content of other elements in the alumina-containing catalyst may be within a range that does not impair the effects in the present disclosure. For example, a content of Fe may be 0.02% by mass or less, or may be zero. The content of Fe in the alumina-containing catalyst may be greater than 0.02% by mass.

The alumina-containing catalyst may also contain an element further other than those listed above. Examples of the element further other elements include Sc, Y, Ti, Zr, Hf, V, Nb, Ta, Cr, Mo, W, Mn, Re, Fe, Ru, Co, Rh, Ir, Ni, Pd, Pt, Cu, Ag, Au, Zn, La, and Ce. An origin of these elements is not limited, and they may be derived from the raw materials or from the manufacturing equipment.

A content of the further other element in the alumina-containing catalyst may be within a range that does not impair the effects in the present disclosure.

Alumina may function not only as a catalyst, but also as a support while functioning as a catalyst. Alumina may also be supported on a support other than alumina.

Examples of the support other than alumina include carbon, zirconia, silica, and titania.

A form of alumina-containing catalyst is not particularly limited and may be powder, pellet, or spherical.

α-alumina is preferably in a molded body such as spheres or pellets, from the viewpoint of excellent filling properties when being filled in a reactor, excellent flowability of reaction gas, and ease of handling in used for around 10 hours.

The molded body differs from a powder and is obtained, for example, by placing powder in a mold and compression-molding it.

A form of the alumina-containing catalyst is not particularly limited and may be powder, pellet, or spherical.

α-alumina is preferably in a molded body such as spheres or pellets, from the viewpoint of excellent filling properties when being filled in a reactor, excellent flowability of reaction gas, and ease of handling in used for around 10 hours.

The molded body differs from a powder and is obtained, for example, by placing powder in a mold and compression-molding it.

### (Reaction Conditions)

In the method of producing a fluoroolefin in the present disclosure, the feed gas need only contain the fluorocarbon represented by Formula (1), and may also contain a component other than the fluorocarbon represented by Formula (1). The feed gas may consist solely of the fluorocarbon represented by Formula (1), or may contain isomers, disproportionation products, impurities, and the like obtained during the production of the fluorocarbon represented by Formula (1). From the viewpoint of suppressing side reactions and catalyst deactivation, the feed gas preferably contains, in addition to the fluorocarbon represented by Formula (1), an inert gas such as nitrogen, argon, helium, carbon dioxide, or octafluorocyclobutane. By an inert gas, the desired product and hydrogen fluoride, which is a by-product, can be diluted. A content of the fluorocarbon represented by Formula (1) with respect to a total amount of the feed gas is preferably 60% by mol or more, more preferably 70% by mol or more, even more preferably 75% by mol or more, and particularly preferably 80% by mol or more.

The method of producing a fluoroolefin in the present disclosure may be carried out in the gas phase or may be carried out in the liquid phase. Because the fluorocarbon represented by Formula (1) is a gas at room temperature, it is preferable to contact the fluorocarbon with the alumina-containing catalyst in the gas phase.

As the reactor for bringing the fluorocarbon into contact with the alumina-containing catalyst, any reactor may be used as long as it can withstand the temperature and pressure described later, and a shape and structure are not particularly limited. Examples of the reactor include a cylindrical vertical reactor. Examples of a material of the reactor include glass, stainless steel, iron, nickel, and alloys primarily composed of iron or nickel. The reactor may be equipped with a heating means, such as an electric heater, for heating the interior of the reactor.

The alumina-containing catalyst may be accommodated in any type of fixed bed, fluidized bed, or moving bed. In the case of a fixed bed, it may be either a horizontal or vertical fixed bed.

The reaction type may be either a flow type or a batch type.

In the fixed bed reactor, various molded bodies of catalyst-supported carriers are filled in order to reduce the pressure loss of the reaction fluid. Similarly to the fixed-bed reactor, a system in which the alumina-containing catalyst is filled, moved by its gravity, and withdrawn from the bottom of the reactor for regeneration is called a moving bed. In a fluidized-bed reactor, in order to operate so that the catalyst layer exhibits characteristics as if it were a fluid by the reaction fluid, the catalyst particles are suspended in the reaction fluid and move within the reactor. A fixed-bed reactor is preferable in that the options for the shape of the alumina-containing catalyst are wide and alumina-containing catalyst wear can be suppressed. As the fixed-bed reactor, there are a tubular reactor and a tank-type reactor, and the tubular reactor is preferable because of ease in controlling the reaction temperature. Furthermore, a multitubular heat-exchange type reactor in which a large number of reaction tubes with a small tube diameter are arranged in parallel and a heat medium is circulated on the outside can be employed. In a case in which a plurality of reactors are provided in series, a plurality of catalyst layers are thereby provided. The catalyst layer may have at least one stage, and may have two or more stages.

From the viewpoint of improving conversion rate, the method of producing a fluoroolefin in the present disclosure is preferably carried out using a flow-through system using a fixed-bed reactor (particularly a vertical fixed-bed reactor).

From the viewpoint of improving industrial productivity, a feed rate of the fluorocarbon to the alumina-containing catalyst (raw material feed rate) is preferably 0.1 g/hr/g or more, more preferably 0.2 g/hr/g or more, and even more preferably 0.3 g/hr/g or more. Furthermore, from the viewpoint of maintaining the conversion rate at a certain level or higher, the raw material feed rate is preferably 5.0 g/hr/g or less, more preferably 4.5 g/hr/g or less, and even more preferably 4.0 g/hr/g or less.

The raw material feed rate refers to an amount of a fluorocarbon (raw material) supplied per gram of alumina-containing catalyst per unit time (g/hr), and it is preferable that an amount of fluorocarbon brought into contact with the catalyst by being supplied thereto is within the above range.

In the method of producing a fluoroolefin in the present disclosure, it is preferable to bring the fluorocarbon into contact with the alumina-containing catalyst at a temperature of from 300 to 800° C., more preferably at a temperature of from 400 to 700° C., and even more preferably at a temperature of from 400 to 600° C. In a case in which the contact temperature is 300° C. or higher, the conversion rate of the fluoroolefin is improved. On the other hand, in a case in which the contact temperature is 800° C. or lower, the decomposition of the fluoroolefin can be suppressed.

In a case in which the contact temperature is 300° C. or higher, the reaction proceeds appropriately. On the other hand, in a case in which the contact temperature is 800° C. or lower, a decrease in selectivity due to cleavage of carbon-carbon bonds of the raw material and a disproportionation reaction of the product (unsaturated compound) are suppressed.

Since the dehydrofluorination reaction is generally an endothermic reaction, maintaining an appropriate reaction temperature can prevent a decrease in conversion rate. As the reaction temperature in the catalyst layer increases, the conversion rate of the raw material increases. Therefore, it is preferable to maintain the reaction temperature in the catalyst layer at a desired temperature to maintain a high conversion rate. One way to maintain the reaction temperature in the catalyst layer at a desired temperature is to extremally heat the catalyst layer using a heat transfer medium or the like. Catalysts typically deteriorate over time as the reaction progresses. Even when a decrease in the conversion rate of the raw material occurs due to catalyst deterioration, the decrease in conversion rate can be prevented by heating the catalyst layer with a heat transfer medium or the like and appropriately maintaining or increasing the reaction temperature. When maintaining or increasing the temperature of the catalyst layer, it is preferable to limit the temperature increase to 50° C. or less to prevent rapid catalyst degradation.

At the start of the reaction, the reaction zone begins at the feed gas inlet. As the catalyst at the feed gas inlet deteriorates over time as the reaction progresses, the reaction zone moves downstream in the gas flow direction. Since the low-temperature product gas generated at the reaction zone flows into a downstream vicinity area of the reaction zone, this downstream vicinity area is usually at the lowest temperature within the catalyst layer. In the present disclosure, the temperature of this lowest-temperature region of the catalyst layer is referred to as the “lowest temperature of the catalyst layer.” The temperature further downstream from the downstream vicinity area usually becomes higher than the lowest temperature of the catalyst layer as it moves away from the reaction zone.

In the method of producing a fluoroolefin in the present disclosure, the feed gas containing a fluorocarbon may be supplied to the reactor at room temperature. However, it is preferable to appropriately heat (preheat) the feed gas before supplying it to the reactor. In a case in which preheating is performed, the feed gas is preferably heated to a temperature of from 80 to 600° C. before being supplied to the reactor. In a case in which preheating is performed at 80° C. or higher, the internal temperature of the reactor is less likely to decrease, and the set conversion rate can be more easily achieved. In a case in which preheating is performed at 600° C. or lower, the internal temperature of the reactor is less likely to increase, undesirable reactions are suppressed, and the selectivity is improved.

The dehydrofluorination reaction in the present disclosure is a reaction in which molecules increase, and therefore, increasing the pressure makes the forward reaction unfavorable.

The pressure when contacting the fluorocarbon with the alumina-containing catalyst is not particularly limited, but from the viewpoint of improving conversion rate, from −0.05 to 2 MPa is preferred, from −0.01 to 1 MPa is more preferred, and from atmospheric pressure to 0.5 MPa is even more preferred.

In the present disclosure, pressure refers to gauge pressure.

A contact time (seconds) between the fluorocarbon and the alumina-containing catalyst is preferably from 0.5 to 100.0 seconds, more preferably from 1.0 to 50.0 seconds, and even more preferably from 2.0 to 20.0 seconds.

The above contact time (seconds) is calculated using the following Formula:

$$Contact time ( seconds ) = [ length of alumina - containing catalyst filled in reactor ( cm ) ] / [ linear velocity ( cm / sec ) ]$$

Linear velocity refers to a speed at which the fluorocarbon passes through the alumina-containing catalyst per unit time.

Furthermore, a contact time (g·sec/mL) between the fluorocarbon and the alumina-containing catalyst is preferably from 1 to 200 g·sec/mL, more preferably from 5 to 175 g·sec/mL, even more preferably from 7 to 150 g·sec/mL, and particularly preferably from 10 to 125 g·sec/mL. In a case in which the contact time (g·sec/mL) is 1 g·sec/mL or more, the conversion rate can be improved. In a case in which the contact time (g·sec/mL) is 200 g·sec/mL or less, equipment cost can be reduced.

The contact time (g·sec/mL) is calculated using the following Formula:

$$Contact time ( g \cdot sec / mL ) = [ Amount of alumina - containing catalyst filled ( g ) ] / [ Fluorocarbon flow rate ( mL / sec ) ]$$

To further reduce the decrease in conversion rate, it is preferable to contact the fluorocarbon with the alumina-containing catalyst in the presence of an inert gas. The inert gas is preferably at least one selected from the group consisting of nitrogen, helium, argon, octafluorocyclobutane, and carbon dioxide. Among them, nitrogen is preferred as the inert gas.

A molar ratio of the fluorocarbon to inert gas in the gas phase is preferably from 0.1 to 30, more preferably from 0.5 to 25.

From the viewpoint of further suppressing a decrease in conversion rate, it is preferable that the fluorocarbon and alumina-containing catalyst be contacted in the gas phase in the presence of water, and that a concentration of the water be less than 500 ppm by mass with respect to a total amount of the feed gas containing a fluorocarbon.

In a case in which a dehydrofluorination reaction is carried out in the gas phase in the presence of water, water adsorbs to the Lewis acid sites on the alumina-containing catalyst surface. It is presumed that, by setting the concentration of water to less than 500 mass ppm with respect to the total amount of the raw material gas containing the fluorocarbon, collapse of Lewis acid sites on the surface of the alumina-containing catalyst, which leads to the formation of structures similar to Bronsted acid sites, is suppressed, and a decrease in the activity of the catalyst is prevented, thereby suppressing a decrease in catalyst activity. It is presumed that, as a result, the conversion rate becomes higher and the desired product can be obtained with high selectivity. The concentration of the water is preferably 300 ppm by mass or less, more preferably 100 ppm by mass or less, even more preferably 50 ppm by mass or less, and particularly preferably 10 ppm by mass or less, in order to further improve the conversion rate and obtain the desired compound with even higher selectivity. Although a lower water concentration is preferable, from the viewpoint that the cost of dehydration treatment of the fluorocarbon and the inert gas and process control become difficult, it is preferably 0.5 ppm by mass or more, and more preferably 1 ppm by mass or more.

When water interacts with the alkali metal or alkaline earth metal in the alumina-containing catalyst, the alumina-containing catalyst becomes alkaline. This changes a structure of the acid sites that indicate dehydrofluorination activity, and it is preferable to prevent a decrease in catalytic activity.

Examples of a common method for measuring the concentration of water include using a commercially available Karl Fischer moisture content meter.

The concentration of the water refers to a content of a water contained in the feed gas when the fluorocarbon is brought into contact with the alumina-containing catalyst. The concentration of the water may also be substituted with a content of a water contained in the feed gas before it is introduced into a reactor.

The fluoroolefin production method in the present disclosure preferably further includes drying the alumina-containing catalyst before contacting the fluorocarbon with the alumina-containing catalyst. Drying the alumina-containing catalyst removes water from the alumina-containing catalyst, increasing its reactivity with fluorocarbons and improving the conversion rate.

A method for drying the alumina-containing catalyst is not particularly limited. The alumina-containing catalyst may be dried before being filled into the reactor or may be dried after being filled into the reactor. Drying the alumina-containing catalyst after filling the reactor is preferred because the reactor can also be preheated together with drying of the alumina-containing catalyst. Specifically, it is preferable to fill the alumina-containing catalyst into the reactor and heat the reactor while flowing an inert gas through it to dry the alumina-containing catalyst.

In the method of producing a fluoroolefin in the present disclosure, hydrogen fluoride is produced as a by-product. Hydrogen fluoride fluorinates oxides contained in the alumina-containing catalyst, strengthening its acidity. Therefore, it is preferable to reduce the hydrogen fluoride concentration. In a case in which the hydrogen fluoride concentration is reduced, the selectivity is maintained, deactivation of the alumina-containing catalyst is suppressed, and a decrease in reaction activity due to a decrease in a specific surface area of the alumina-containing catalyst is suppressed.

One method for reducing the hydrogen fluoride concentration is to dilute it with an inert gas. Since the use of an inert gas increases the energy load of the purification process after the reaction, it is preferable that the use of the inert gas be appropriately controlled.

It is preferable that the hydrogen fluoride concentration during the reaction be 15% by mol or less. From the viewpoint of extending the catalyst life, the hydrogen fluoride concentration is more preferably 13% by mol or less, even more preferably 10% by mol or less, particularly preferably 8% by mol or less, and most preferably 7% by mol or less. Furthermore, from the viewpoint of productivity and the energy load of the purification process, the hydrogen fluoride concentration is preferably 0.5% by mol or more, more preferably 0.8% by mol or more, even more preferably 1.0% by mol or more, particularly preferably 1.3% by mol or more, and most preferably 1.5% by mol or more.

The method of producing a fluoroolefin in the present disclosure is preferably carried out in the presence of an oxidizing agent. From the viewpoint that the conversion rate is high and the desired compound can be obtained with high selectivity, the oxidizing agent is preferably oxygen, chlorine, bromine, or iodine. Among them, oxygen is more preferred as the oxidizing agent. In the present disclosure, a concentration of the oxidizing agent is preferably from 0.01 to 21% by mol with respect to the feed gas. The concentration of the oxidizing agent is more preferably from 1 to 20% by mol, even more preferably from 5 to 18% by mol, and particularly preferably from 7.5 to 16% by mol, with respect to the raw material compound, from the viewpoint of further improving the conversion rate and obtaining the desired compound with even higher selectivity.

In the present disclosure, the conversion rate refers to a ratio (mol %) of a total molar amount of compounds other than the raw material compound contained in the effluent gas from the reactor outlet to a molar amount of the raw material compound supplied to the reactor.

In general, a higher conversion rate is preferable from the viewpoint of productivity. However, in the dehydrofluorination reaction in the present disclosure, it is preferable to appropriately control the conversion rate. Controlling the conversion rate reduces the concentration of hydrogen fluoride produced in the gas phase, which is thought to suppress alumina-containing catalyst deactivation by hydrogen fluoride. While diluting the gas phase with an inert gas is one possible method, using an inert gas increases the energy load in the purification process after the reaction, and therefore, using excessive inert gas is not preferable. From the viewpoint of extending the catalyst life, it is preferable to keep the hydrogen fluoride concentration during the reaction to 15% by mol or less.

In particular, in the method of producing a fluoroolefin in the present disclosure, by using HFC-134a as the fluorocarbon, the conversion rate does not become too high and deactivation of the alumina-containing catalyst deactivation is suppressed.

In light of the above, a conversion rate 10 hours after contacting the fluorocarbon with the catalyst is preferably from 5.0 to 15.0%, more preferably from 7.0 to 13.0%, and even more preferably from 5.0 to 12.5%.

In the present disclosure, selectivity refers to a ratio (mol %) of a molar amount of the desired product contained in the reactor outlet gas to a total molar amount of compounds other than the raw material compound contained in the reactor outlet gas.

A selectivity of 100% is preferable because it eliminates the need for a post-reaction purification process, but side reactions may occur within the reaction temperature range required to achieve a desired conversion rate. A higher selectivity is preferable because it reduces an amount of waste, the energy load of the post-reaction purification process, and extends the catalyst life.

A selectivity 10 hours after contacting the fluorocarbon with the alumina-containing catalyst is preferably 90% or higher, more preferably 93% or higher, and even more preferably 95% or higher.

In particular, in the method of producing a fluoroolefin in the present disclosure, HFO-1123 can be obtained with high selectivity by using HFC-134a as the fluorocarbon.

Examples of a compound other than the raw material compound and the desired product contained in the reactor outlet gas include hydrogen fluoride, carbon monoxide, carbon dioxide, and water. For example, in a case in which HFC-134a is used as the fluorocarbon raw material compound, the other compound may include HFC-134, 1,1-difluoroethylene (VdF), E/Z-1,2-difluoroethylene (HFO-1132(E)/(Z)), and the like.

### EXAMPLES

Hereinafter, the present disclosure will be described more specifically by Examples; however, the present disclosure is not limited to the following Examples as long as the gist thereof is not exceeded.

Examples 1 to 9 are examples, and Examples 10 to 13 are comparative examples.

### Example 1

α-alumina (product name “N612N,” manufactured by JGC Catalysts and Chemicals Ltd) was weighed in an amount of 1 mL and used as a catalyst. A stainless steel (SUS304) reactor tube with an inner diameter of 10 mm and a length of 30 cm was filled with the catalyst and placed in a tubular electric furnace. The catalyst-filled section was heated to 475° C. in the furnace while nitrogen was circulating, dehydrating the catalyst. A nitrogen/HFC-134a (0.1/1 mol/mol) mixed gas was then passed through the tube for a contact time of 4.7 seconds to carry out the dehydrogenation reaction to HFO-1123.

A water concentration in the nitrogen/HFC-134a (0.1/1 mol/mol) mixed gas was measured using a Karl Fischer moisture content analyzer and found to be 5 ppm by mass.

### Examples 2 to 13

In Examples 2 to 13, except that the catalyst was changed and the various conditions were changed to the values shown in Table 2, the dehydrofluorination reaction was carried out in the same manner as in Example 1.

Hereinafter, the catalysts used in Examples 2 to 13 will be described.

### Example 2

γ-alumina (product name “N612N”, manufactured by JGC Catalysts and Chemicals Ltd) was pulverized with a mortar to obtain a powder. The resulting powder was calcined at 1,300° C. for 6 hours under an air atmosphere And then, it was analyzed by X-ray diffraction and found to be primarily composed of α-alumina. This powder was used as a catalyst.

### Example 3

α-alumina (product name “SA52124”, manufactured by Saint-Gobain) was used as the catalyst.

### Example 4

α-alumina (product name “SA52238”, manufactured by Saint-Gobain) was used as the catalyst.

### Example 5

α-alumina (product name “FGL-30”, manufactured by Iwatani Chemical Industries, Ltd.) was used as the catalyst.

### Example 6

α-alumina (product name “FGL-40”, manufactured by Iwatani Chemical Industries, Ltd.) was used as the catalyst.

### Example 7

α-alumina (product name “C500”, manufactured by Nippon Light Metal Co., Ltd.) was used as the catalyst.

### Example 8

α-alumina (product name “LT303D”, manufactured by Nippon Light Metal Co., Ltd.) was used as the catalyst.

### Example 9

α-alumina (product name “AKQ-10”, manufactured by Sumitomo Chemical Co., Ltd.) was used as the catalyst.

### Example 10

α-alumina (product name “SA51161”, manufactured by Saint-Gobain) was used as the catalyst.

### Example 11

α-alumina (product name “SA5561”, manufactured by Saint-Gobain) was used as the catalyst.

### Example 12

α-alumina (product name “SA5218”, manufactured by Saint-Gobain) was used as the catalyst.

### Example 13

α-alumina (product name “SA5252”, manufactured by Saint-Gobain) was used as the catalyst.

Elemental analysis of each catalyst was performed using a ZSX Primus II scanning X-ray fluorescence analyzer manufactured by Rigaku Corporation under the following conditions: X-ray output: 50 kV, 72 mA, measurement area: 20 mmφ, measurement time: 30 minutes. The results of the elemental analysis of the catalysts used in Examples 1 to 13 are shown in Table 1. The values in the table are expressed in % by mass, and a blank entry indicates that the corresponding element was below the detection limit.

TABLE 1

| | Example | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| B | | | | | | | | | | | | | |
| N | 0.39 | 0.39 | | | | | | | | | | | |
| C | 5.4 | 5.4 | 0.96 | 1.39 | 0.86 | 0.91 | 2.2 | 1.4 | 0.77 | 0.67 | 1.2 | 1.1 | 0.90 |
| O | 49.4 | 49.4 | 49.4 | 49.5 | 49.6 | 49.3 | 48.2 | 48.5 | 48.8 | 49.8 | 49.3 | 47.7 | 48.2 |
| F | | | | | | | | | | | | | |
| Na | | | | | | | | | | | | 0.58 | |
| Mg | | | | | | | | | | | 0.050 | 0.42 | 0.15 |
| Al | 44.8 | 44.8 | 49.6 | 49.1 | 49.6 | 49.7 | 49.6 | 50.1 | 50.4 | 49.2 | 49.0 | 31.5 | 47.2 |
| Si | 0.0174 | 0.0174 | 0.021 | 0.026 | | 0.056 | 0.030 | | | 0.21 | 0.38 | 15.6 | 3.0 |
| S | 0.0188 | 0.0188 | | | | | | | | | | | |
| Cl | | | | | | | | | | | | | |
| Ca | | | | | | | | | | 0.037 | | 1.07 | 0.28 |
| Cr | | | | | | | | | | | | | |
| Fe | 0.0096 | 0.0096 | | | | | | | | 0.042 | 0.036 | 0.29 | 0.17 |
| Ni | | | | | | | | | | | | | |
| Zn | 0.0014 | 0.0014 | 0.0086 | | | | | | | | | | |
| Nb | | | | | | | | | | | | | |
| Y | | | | | | | | | | | | | |
| Mo | | | | | | | | | | | | | |
| Eu | | | | | | | | | | | | | |
| Re | | | | | | | | | | | | | |
| K | | | | | | | | | | | 0.042 | 1.61 | 0.073 |
| P | | | | | | | | | | 0.028 | 0.011 | 0.0389 | 0.0080 |
| Ti | | | | | | | | | | | | | |
| As | | | | | | | | | | | | | |
| Zr | | | | | | | | | | | 0.0032 | | 0.0049 |
| Ga | 0.0056 | 0.0056 | | | | | 0.025 | | | 0.0096 | 0.0059 | 0.011 | 0.0074 |
| Mn | | | | | | | | | | | | | |
| V | | | | | | | | | | | | | |
| Cu | | | | | | | | | | | | | |
| Ba | | | | | | | | | | | | | |
| Hf | | | | | | | | | | | | | |
| Rb | | | | | | | | | | | | | |
| Sr | | | | | | | | | | | | 0.0071 | |
| Pd | | | | | | | | | | | | | |

A contact time (seconds) was calculated using the following Formula:

$$Contact time ( seconds ) = [ length of catalyst filled in the reactor ( cm ) ] / [ linear velocity ( cm / sec ) ]$$

Linear velocity refers to a speed at which the fluorocarbon passes through the catalyst per unit time.

A contact time (g-min/mL) was calculated using the following Formula:

$$Contact time ( g min / mL ) = [ Amount of Catalyst filled ( g ) ] / [ Fluorocarbon flow rate ( mL / min ) ]$$

In the reactions of Examples 1 to 13, the product gas (hereinafter also referred to as “reactor outlet gas”) extracted from the reactor outlet 10 hours after the start of the reaction was analyzed using a gas chromatograph. Specifically, a gas chromatograph (product name “GC6850” manufactured by Agilent) was equipped with a column (product name “DB-1301” manufactured by Agilent, length 60 m, inner diameter 0.25 mm, film thickness 1 μm) for analysis. A molar amount calculated from an area ratio (GCArea %) of the reactor outlet gas was used to calculate the conversion rate of HFC-134a and the selectivity of HFO-1123.

### (Conversion Rate of HFC-134a)

This refers to a ratio (mol %) of a total molar amount (M1) of components other than HFC-134a contained in the reactor outlet gas to a total molar amount (M₁₃₄ₐ) of HFC-134a fed to the reactor.

$$Converstion Rate of HFC - 134 a ( \% ) = ( M 1 / {M}_{134 a} ) \times 100$$

### (Selectivity of HFO-1123)

This refers to a ratio (mol %) of a molar amount (M₁₁₂₃) of HFO-1123 contained in the reactor outlet gas to the total molar amount (M1) of compounds other than HFC-134a contained in the reactor outlet gas.

$$Selectivity of HFO - 1123 ( \% ) = ( {M}_{1123} / M 1 ) \times 100$$

TABLE 2

| | | Example | | | | | | |
|---|---|---|---|---|---|---|---|---|
| | | 1 | 2 | 3 | 4 | 5 | 6 | 7 |
| Catalyst | Alkali Metal A | 0 | 0 | 0 | 0 | 0 | 0 | 0 |
| [ppm by | Alkaline Earth | 0 | 0 | 0 | 0 | 0 | 0 | 0 |
| mass] | metal B | | | | | | | |
| | A + B | 0 | 0 | 0 | 0 | 0 | 0 | 0 |
| | Si | 174 | 174 | 214 | 260 | 0 | 556 | 295 |

| | | | | | | | |
|---|---|---|---|---|---|---|---|
| Contact Temperature [° C.] | 450 | 450 | 450 | 450 | 450 | 450 | 450 |
| Pressure [MPaG] | 0 | 0 | 0 | 0 | 0 | 0 | 0 |
| Molar Ratio: | 10 | 10 | 10 | 10 | 10 | 10 | 10 |
| Raw Material/N₂ [mol/mol] | | | | | | | |
| Contact Time [sec] | 4.7 | 4.6 | 4.7 | 4.7 | 4.7 | 4.7 | 4.7 |
| Contact Time [g · sec/mL] | 0.20 | 0.21 | 0.23 | 0.17 | 0.26 | 0.28 | 0.09 |
| Raw Material Feed Rate | 1.25 | 1.19 | 1.09 | 1.45 | 0.96 | 0.89 | 3.08 |
| [g/hr/g] | | | | | | | |
| Conversion Rate [%] | 8.4 | 10 | 11.8 | 12.1 | 8.1 | 11.5 | 10.1 |
| Selectivity [%] | 97 | 99 | 99 | 99 | 100 | 98 | 98 |

| | | Example | | | | | |
|---|---|---|---|---|---|---|---|
| | | 8 | 9 | 10 | 11 | 12 | 13 |
| Catalyst | Alkali Metal A | 0 | 0 | 0 | 424 | 21826 | 733 |
| [ppm by | Alkaline Earth | 0 | 0 | 370 | 500 | 14891 | 4299 |
| mass] | metal B | | | | | | |
| | A + B | 0 | 0 | 370 | 924 | 36717 | 5032 |
| | Si | 0 | 0 | 2085 | 3750 | 156369 | 29824 |

| | | | | | | |
|---|---|---|---|---|---|---|
| Contact Temperature [° C.] | 450 | 450 | 450 | 450 | 450 | 450 |
| Pressure [MPaG] | 0 | 0 | 0 | 0 | 0 | 0 |
| Molar Ratio: | 10 | 10 | 10 | 10 | 10 | 10 |
| Raw Material/N₂ [mol/mol] | | | | | | |
| Contact Time [sec] | 4.7 | 4.7 | 4.7 | 4.7 | 4.7 | 4.7 |
| Contact Time [g · sec/mL] | 0.35 | 0.19 | 0.24 | 0.22 | 0.35 | 0.29 |
| Raw Material Feed Rate | 0.79 | 1.45 | 1.02 | 1.11 | 0.71 | 0.86 |
| [g/hr/g] | | | | | | |
| Conversion Rate [%] | 10.3 | 9.0 | 0.5 | 1.0 | 0.2 | 0.3 |
| Selectivity [%] | 100 | 97 | 75 | 19 | 61 | 35 |

As shown in Table 2, Examples 1 to 9 include contacting a fluorocarbon represented by the following Formula (1) with an alumina-containing catalyst to produce a fluoroolefin represented by the following Formula (2), in which the alumina-containing catalyst satisfies at least one of (I) a total content of alkali metal elements and alkaline earth metal elements is 100 ppm by mass or less; or (II) a Si content is 1000 ppm by mass or less. This resulted in a higher conversion rate than conventional methods.

On the other hand, in Examples 10 to 13, which satisfied neither condition (I) nor (II), the conversion rate was significantly lower as compared with Examples 1 to 9. In addition, in Examples 10 to 13, the selectivity was also significantly lower as compared with Examples 1 to 9.

The disclosure of Japanese Patent Application No. 2023-072721 is incorporated herein by reference in its entirety. All literature, patent applications, and technical standards described herein are incorporated herein by reference to the same extent as if each individual literature, patent application, and technical standard were specifically and individually indicated to be incorporated by reference.

1. A method of producing a fluoroolefin, the method comprising contacting a fluorocarbon represented by the following Formula (1) with an alumina-containing catalyst to produce a fluoroolefin represented by the following Formula (2), 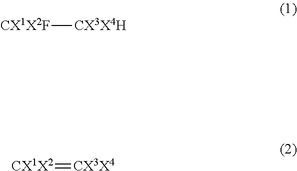
wherein, in Formulas (1) and (2), X¹, X², X³ and X⁴ each independently represent a hydrogen atom or a fluorine atom, provided that at least one of X¹, X², X³ or X⁴ is a fluorine atom, and
wherein the alumina-containing catalyst satisfies at least one of the following (I) or (II):
(I) a total content of alkali metal elements and alkaline earth metal elements is 100 ppm by mass or less;
(II) a Si content is 1000 ppm by mass or less.

2. The method of producing a fluoroolefin according to claim 1, wherein the alumina contained in the alumina-containing catalyst includes α-alumina.

3. The method of producing a fluoroolefin according to claim 1, wherein the fluorocarbon is at least one selected from the group consisting of 1,1,1-trifluoroethane, 1,1,2-trifluoroethane, 1,1,2,2-tetrafluoroethane, and 1,1,1,2-tetrafluoroethane.

4. The method of producing a fluoroolefin according to claim 1, wherein the fluoroolefin is at least one selected from the group consisting of 1,2-difluoroethylene, 1,1-difluoroethylene, and trifluoroethylene.

5. The method of producing a fluoroolefin according to claim 1, wherein the fluorocarbon is 1,1,1,2-tetrafluoroethane, and the fluoroolefin is trifluoroethylene.

6. The method of producing a fluoroolefin according to claim 1, wherein the fluorocarbon and the alumina-containing catalyst are contacted at a temperature of from 300 to 800° C.

7. The method of producing a fluoroolefin according to claim 1, wherein:
the fluorocarbon and the alumina-containing catalyst are contacted in the presence of an inert gas; and
the inert gas is at least one selected from the group consisting of nitrogen, helium, argon, octafluorocyclobutane, and carbon dioxide.

8. The method of producing a fluoroolefin according to claim 1, further comprising drying the alumina-containing catalyst before contacting the fluorocarbon with the alumina-containing catalyst.

9. The method of producing fluoroolefins according to claim 1, wherein:
the fluorocarbon and the alumina-containing catalyst are contacted in a gas phase in the presence of water; and
a concentration of the water is less than 500 ppm by mass with respect to a total amount of a feed gas containing the fluorocarbon.
